(19) United States
(12) Patent Application Publication
(10) Pub. No.: US 2015/0150503 A1
(43) Pub. Date: Jun. 4, 2015

(54) APPARATUS, SYSTEMS, AND METHODS FOR MONITORING EXTRAVASCULAR LUNG WATER

(71) Applicant: THE BOARD OF TRUSTEES OF THE LELAND STANFORD JUNIOR UNIVERSITY, Palo Alto, CA (US)

(72) Inventors: Ravinder D. Pamnani, Belmont, CA (US); Sidhartha R. Sinha, Menlo Park, CA (US); Joelle K. Barral, Mountain View, CA (US); Michael P. Schaller, Redwood City, CA (US); Paul G. Yock, Atherton, CA (US); Anshuman Bhuyan, San Jose, CA (US); Pierre Khuri-Yakub, Palo Alto, CA (US); Roman Solek, Pleasanton, CA (US)

(21) Appl. No.: 14/556,056

(22) Filed: Nov. 28, 2014

Related U.S. Application Data
(63) Continuation of application No. PCT/US13/43195, filed on May 29, 2013.
(60) Provisional application No. 61/801,739, filed on Mar. 15, 2013.
(60) Provisional application No. 61/652,521, filed on May 29, 2012.

(51) Int. Cl. A61B 5/00; A61B 8/00; A61B 5/08; A61B 8/08

(52) U.S. Cl. CPC A61B 5/4878; A61B 8/08; A61B 8/4236; A61B 8/4281; A61B 8/4494; A61B 8/5223; A61B 8/543; A61B 5/08; A61B 8/56

(57) ABSTRACT

Apparatus, systems, and methods are provided for monitoring the extravascular lung water status of a patient. For example, an acoustic diagnostic device is provided that includes a housing configured to be worn by a patient and including a patient contact surface configured to contact the patient's skin of the patient's thorax; an acoustic transducer for transmitting acoustic energy via the patient contact surface into the patient's thorax and receiving reflected acoustic energy from the patient's thorax; and one or more processors coupled to the acoustic transducer for analyzing the reflected acoustic energy to provide an indication of extravascular lung water status of the patient.

### RELATED APPLICATION DATA

This application is a continuation of co-pending International Application No. PCT/US2013/043195, which claims benefit of U.S. provisional application Ser. No. 61/652,521, filed May 29, 2012, and 61/801,739, filed Mar. 15, 2013, the entire disclosures of which are expressly incorporated by reference herein.

### FIELD OF THE INVENTION

The present invention relates generally to apparatus, systems, and methods for monitoring extravascular lung water status of patients, and more particularly, to wearable and/or disposable devices and methods for determining and/or otherwise monitoring extravascular lung water status of patients.

### BACKGROUND

Heart failure is a complex disease affecting almost six million Americans. The disease is growing with six hundred thousand (600,000) new patients diagnosed annually. While heart failure has multiple causes, a primary treatment path is a restoration of fluid balances such as through the use of diuretics. Exacerbation is typically the result of pulmonary edema, but it is difficult to predict because the underlying fluid imbalances often start discreetly, days before symptoms (dyspnea, fatigue, etc.) that may lead to hospital admission.

According to some studies, hospitalization costs for heart failure exceed twenty billion dollars a year, rehospitalizations represent five to ten billion dollars of these expenses, and fluid management is the driver of at least eight percent (80%) of these rehospitalizations.

Ultrasound B-lines, also called “lung comets,” are a measure of the presence of extravascular lung water (pulmonary edema) and may be observed in diseased lungs. Ultrasound B-lines exist when acoustic energy is transmitted into the thorax and then reflects back and forth within the intralobular spaces due to the presence of extravascular lung water. They appear as solid white streaks on ultrasound monitors and are clearly distinguishable from other acoustic reflections. Their presence may be used in dyspneic patients presenting in the intensive care unit or emergency department to differentiate between pneumonia and heart failure with pulmonary edema. Ultrasound B-lines have been included in recent guidelines by the American College of Chest Physicians as an indication for alveolar interstitial pattern. Intra- and inter-observer variability of counting ultrasound B-lines to determine a “lung comets score” have been shown to be less than five percent (5%), making ultrasound B-lines more consistent than chest X-ray for assessing pulmonary edema.

Ultrasound devices currently used for the assessment of ultrasound B-lines in hospital settings are systems typically including a piezoelectric probe connected through a cable to a processing unit and monitor. These are large and bulky systems and, as such, they are not ideal for mobile usage and continuous monitoring of a patient. Additionally, current ultrasound systems generally require the expertise of a trained professional such as a physician or a nurse to find and locate the ultrasound B-lines and are therefore not easily used by patients. Further, the assessment of the ultrasound data collected by the user requires analysis and interpretation by a trained professional.

Implantable devices have also been suggested that use acoustic energy for monitoring the fluid status in the lungs of a patient with heart failure using ultrasound B-lines. Because these devices are intended to be implanted, they require a surgical procedure before they may be implemented. Therefore, the invasiveness of such implantable devices may be a barrier to adoption.

Accordingly, devices capable of monitoring the extravascular lung water status of a patient through the use of ultrasound B-lines would be useful to physicians and patients and could be potentially utilized as an early detection of pulmonary edema before other clinical symptoms present themselves.

### SUMMARY

The present invention is directed to apparatus, systems, and methods for monitoring extravascular lung water status of patients. More particularly, the present invention is directed to wearable and/or disposable devices, e.g., externally fixated to the skin of a patient, for determining and/or otherwise monitoring extravascular lung water status of patients over a period of time through the use of acoustic energy, and to systems and methods that include such devices.

In accordance with a first embodiment, an acoustic diagnostic device is provided that includes a housing configured to be worn by a patient and including a patient contact surface configured to contact the patient's skin of the patient's thorax; an acoustic transducer for transmitting acoustic energy via the patient contact surface into the patient's thorax and receiving reflected acoustic energy from the patient's thorax; and one or more processors coupled to the acoustic transducer for analyzing the reflected acoustic energy to provide an indication of extravascular lung water status of the patient.

In one embodiment, the patient contact surface may include a periphery surrounding a gel reservoir, the periphery including an adhesive region to substantially fix the housing relative to the patient's skin, and acoustic coupling material in the gel reservoir for acoustically coupling the acoustic transducer to the patient's skin.

In exemplary embodiments, the acoustic transducer may include a single or multiple transducer elements, e.g., a linear array of transducer elements. The transducer element(s) may be piezoelectric transducer elements, a capacitive micromachined ultrasonic transducer, or a single crystal transducer element.

The one or more processors may be configured to analyze the reflected acoustic energy to determine one or more of a number of the responsive acoustic echoes per unit of time, an intensity, and a frequency of the reflected acoustic energy. For example, the one or more processors may be configured to intermittently activate the acoustic transducer to transmit acoustic energy via the patient contact surface into the patient's thorax and receive reflected acoustic energy from the patient's thorax, and the one or more processors analyze the reflected acoustic energy to determine changes in the extravascular lung water status of the patient over time.

Optionally, the device may include a communication interface, e.g., a wireless transmitter, coupled to the one or more processors for communicating information regarding the extravascular lung water status of the patient to a remote location.

In another option, the device may include a motion sensor coupled to the one or more processors, and the one or more processors may be configured to acquire motion data from the motion sensor to determine an activity status of the patient. For example, the one or more processors may activate the acoustic transducer only when a predetermined activity status of the patient is confirmed.

In still another option, the device may include an output device coupled to the one or more processors for providing an indication of the extravascular lung water status of the patient, e.g., one or more of a display, a set of indicator lights, and a speaker.

In accordance with another embodiment, a system is provided for monitoring extravascular lung water of a patient that includes an acoustic diagnostic device (or optionally multiple devices), and a base station for receiving the information from the acoustic diagnostic device regarding the extravascular lung water status of the patient. In an exemplary embodiment, the acoustic diagnostic device may include a housing configured to be worn by a patient and including a patient contact surface configured to contact the patient's skin of the patient's thorax; an acoustic transducer for transmitting acoustic energy via the patient contact surface into the patient's thorax and receiving reflected acoustic energy from the patient's thorax; one or more processors coupled to the acoustic transducer for processing the reflected acoustic energy; and a communication interface for communicating information regarding the reflected acoustic energy to a remote location. The base station may receive the information via the communication interface, and monitor the extravascular lung water status of the patient based at least in part on the information.

Optionally, the base station may include an output device for providing an indication of the extravascular lung water status of the patient. In addition or alternatively, the base station may include a network interface for communicating data regarding the extravascular lung status of the patient to a remote location, e.g., to a clinician or other caregiver.

In an exemplary embodiment, the base station may include a processor configured to analyze the information regarding the reflected acoustic energy, e.g., to determine a number of the responsive acoustic echoes per unit of time, to determine at least one of an intensity and a frequency of the reflected acoustic energy, and the like. Optionally, the communication interface may include a receiver for receiving instructions from the base station, and the base station may be configured to send instructions to the acoustic diagnostic device, e.g., to intermittently activate the acoustic transducer to transmit acoustic energy and receive reflected acoustic energy from the patient's thorax. The base station may be configured to analyze the reflected acoustic energy to determine changes in the extravascular lung water status of the patient over time.

In accordance with yet another embodiments, a method is provided for monitoring extravascular lung water of a patient that includes fixing an acoustic transducer device relative to the patient's skin of the patient's thorax; activating the acoustic transducer device to transmit acoustic energy into the patient's thorax and receive reflected acoustic energy from the patient's thorax; and analyzing the reflected acoustic energy to monitor the extravascular lung water status of the patient.

In accordance with still another embodiment, a diagnostic device is provided that includes an adhesive region capable of fixating to a skin of a thorax region; an acoustic transducer assembly adjacent to the adhesive region and capable of transmitting acoustic energy to a lung and receiving responsive acoustic echoes from the lung; one or more circuits capable of receiving acoustic information from the acoustic transducer assembly and performing calculations based at least in part on the responsive acoustic echoes; and a power supply capable of energizing the acoustic transducer assembly and the one or more circuits, wherein the one or more circuits are configured to analyze the received acoustic information to provide an indication of an extravascular lung water status.

In accordance with another embodiment, a diagnostic device is provided that includes a band capable of encircling a thorax region; an acoustic transducer assembly carried by the band and capable of transmitting acoustic energy to a lung and receiving responsive acoustic echoes from the lung; one or more circuits capable of receiving acoustic information from the acoustic transducer assembly and performing calculations based at least in part on the responsive acoustic echoes; and a power supply capable of energizing the acoustic transducer assembly and the one or more circuits, wherein the one or more circuits are configured to analyze the received acoustic information to provide an indication of an extravascular lung water status.

In accordance with still another embodiment, a diagnostic system is provided that includes a) a first device including an adhesive region capable of fixating to a skin of a thorax region; an acoustic transducer assembly adjacent to the adhesive region and capable of transmitting acoustic energy towards a lung within the thorax region; and a power supply capable of energizing the acoustic transducer assembly, and b) a second device including an adhesive region capable of fixating to a skin of a thorax region; an acoustic transducer assembly adjacent to the adhesive region and capable of receiving acoustic energy reflected from the lung; one or more circuits capable of receiving acoustic information from the acoustic transducer assembly and performing calculations; and a power supply capable of energizing the acoustic transducer assembly and the one or more circuits. The first device and the second device may be configured to be fixated to the skin of a thorax region such that the acoustic energy transmitted from the acoustic transducer assembly of the first device is received by the acoustic transducer assembly of the second device, whereby the one or more circuits of the second device are configured to analyze the received acoustic energy to provide an indication of an extravascular lung water status.

In accordance with yet another embodiment, a method is provided that includes emitting acoustic energy toward a lung using a device fixated to a skin of a thorax region of a person; receiving and processing one or more acoustic energy echoes; and calculating and providing an indication of extravascular lung water status.

Other aspects and features of the present invention will become apparent from consideration of the following description taken in conjunction with the accompanying drawings.

### BRIEF DESCRIPTION OF THE DRAWINGS

It will be appreciated that the exemplary apparatus shown in the drawings are not necessarily drawn to scale, with emphasis instead being placed on illustrating the various aspects and features of the illustrated embodiments.

FIG. 1A is a perspective view of an exemplary embodiment of a wearable acoustic diagnostic device.

FIG. 1B is a schematic showing an exemplary embodiment of an acoustic diagnostic device including several optional components.

FIG. 2A is a cross-sectional view of the device of FIG. 1A.

FIG. 2B is a cross-sectional view of an alternative embodiment of a wearable acoustic diagnostic device.

FIG. 3 is a schematic view showing an exemplary array of acoustic transducer elements that may be provided in a wearable acoustic diagnostic device transmitting acoustic energy and/or receiving acoustic energy echoes.

FIG. 4 is a cross-sectional view of an acoustic diagnostic device, such as the device of FIG. 1A, placed on a thorax of a patient and in operation transmitting and receiving acoustic energy.

FIG. 5 is an exemplary ultrasound image that may be obtained using an acoustic diagnostic device, such as that shown in FIG. 1A.

FIG. 6 is a graph showing an exemplary output of RF-Signal phase information that may be produced using an acoustic diagnostic device, such as that shown in FIG. 1A.

FIG. 7 shows a system including a base station and a plurality of acoustic diagnostic devices worn by a patient and communicating with the base station.

### DETAILED DESCRIPTION OF THE EXEMPLARY EMBODIMENTS

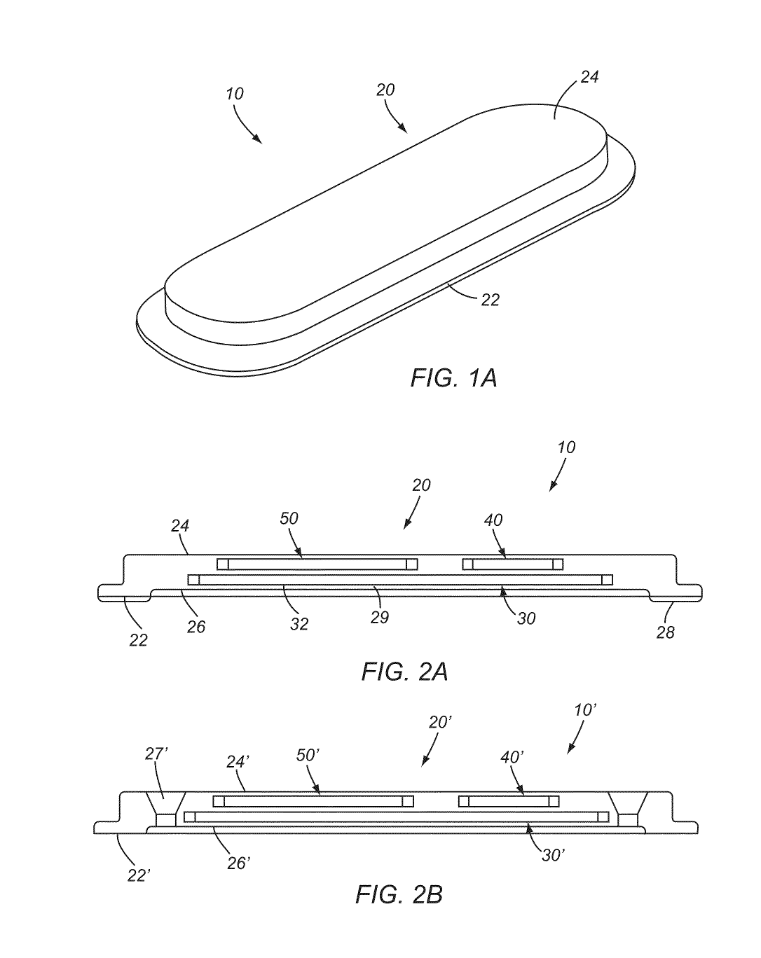

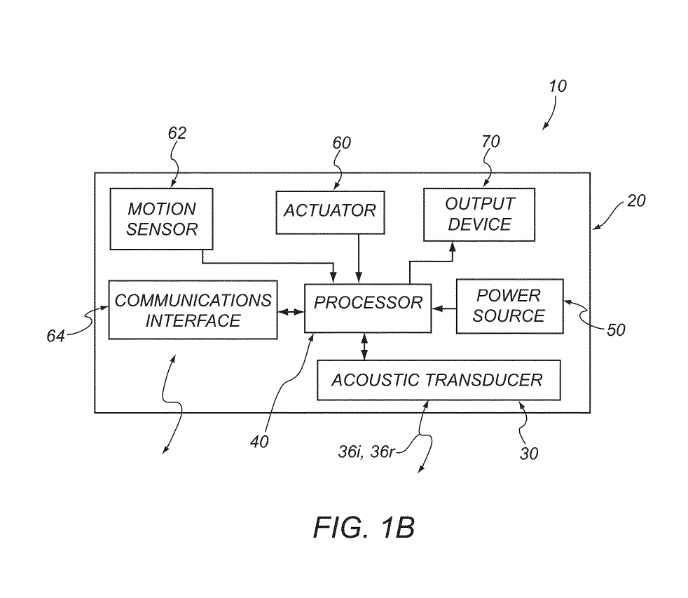

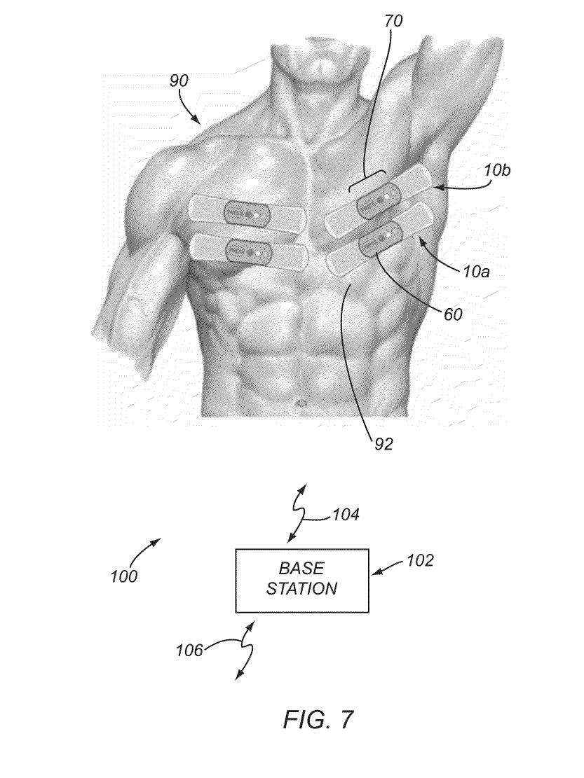

Turning to the drawings, FIGS. 1A and 2A show an exemplary embodiment of a wearable or external acoustic diagnostic device 10 that includes a housing or encasement material 20 carrying components of the device 10, including one or more acoustic transducers 30, processors 40, power sources 50, and the like, e.g., to monitor the extravascular lung water status of a patient (not shown). Optionally, as shown in FIG. 1B, the device 10 may include one or more additional components, e.g., within the housing 20, such as one or more actuators 60, motion sensors 62, a communication interface 64, an output device 70, and the like (not shown), a strap or band (also not shown) to secure the device 10 to a patient's body, and the like, as described elsewhere herein. In one embodiment, the device 10 may be configured as a relatively small, flat patch, which may be sufficiently lightweight and/or otherwise unobtrusive to be worn by a patient, e.g., secured directly to the patient's skin, with minimal discomfort and/or inconvenience. In addition or alternatively, the device 10 may be included as part of a system 100 for monitoring a patient, e.g., including a remote base station 102 communicating with the device 10 and/or other components, as shown in FIG. 7 and described elsewhere herein. In another alternative, the device 10 may be a handheld device, e.g., usable by a patient, clinician, or other caregiver, which may be held against the patient's body only when monitoring is desired.

Generally, as shown in FIGS. 1A and 2A, the housing 20 surrounds and/or encompasses the internal components 30-50, to provide a sealed, e.g., substantially fluid tight, enclosure to protect the internal components 30-50. The housing 20 may include a substantially flat, concave, or otherwise shaped patient contact surface 22, e.g., for placement against the skin of a patient, and an outer or back surface 24 opposite and attached to the patient contact surface 22 to enclose the internal components.

The housing 20 may be formed from soft and/or flexible material such as silicone, low durometer polyurethane, and/or other suitable material, e.g. capable of conforming to the thorax of a patient and/or providing electrical and/or thermal insulation of the internal components of the device 10. Alternatively, the housing 20 may be formed from multiple materials, e.g., such that the housing 20 is softer and/or more comfortable along the patient contact surface 22 while other sections, such as the outer or back surface 24 of the housing 20, may be more rigid, e.g., to protect the internal components 30-50 from damage, while still allowing the device 10 to be applied to a patient's skin with substantial contact by the patient contact surface 22.

As can be seen in FIG. 2A, the patient contact surface 22 may include a gel reservoir 26, for example, one or more recesses on the underside of the device 10. As shown, the gel reservoir 26 may be an elongate (e.g., elliptical, similar to the housing 20 shown in FIG. 1) recess surrounded by a raised periphery 22 that may contact the patient's skin directly. In an exemplary embodiment, the gel reservoir 26 may be between about 0.5 to 3.1 millimeters (0.020-0.125 inch) deep. Thus, the bottom of the gel reservoir 26 may provide an open area that faces the skin of the patient, which may be filled with gel or other acoustic coupling material, e.g., substantially even with or thicker than the periphery of the patient contact surface 22, to enhance acoustic coupling, fixation, and/or other contact between the device 10 and the patient's skin.

Optionally, as shown in FIG. 2B, a device 10′ (otherwise similar to the device 10 and/or other devices herein) may be provided in which the housing 20′ has one or more injection holes 27′ (e.g., two as shown) that communicate between the outer surface 24′ and the gel reservoir 26,′ e.g., allowing gel or other coupling material to be injected into the gel reservoir 26′ from the outside of the device 10,′ e.g., before or after being placed on a patient's skin. If desired, the injection holes 27′ may be connected to a pump and/or separate gel container (not shown), which may allow manual or automated, e.g., intermittent or substantially continuous, injection of coupling material into the gel reservoir 27,′ e.g., while the device 10′ is worn by a patient. Optionally, a source of vacuum (not shown) may be coupled to the injection holes 27,′ e.g., to create a vacuum within the gel reservoir 26′ after placing the device 10′ against a patient's skin, which may enhance fixation of the device 10 relative to the patient.

In addition or alternatively, at least a portion of the patient contact surface 22 may include adhesive, tacky, or other material, e.g., to enhance fixation relative to the patient's skin. For example, as shown in FIG. 2A, the periphery of the patient contact surface 22 surrounding the gel reservoir 26 may include an adhesive region 28 that may contact the skin when the device 10 is worn by the patient. The adhesive region 28 may include a biocompatible adhesive, for example, a hydrocolloid adhesive capable of releasably adhering the device 10 to the skin, e.g. for between about three and fourteen (3-14) days or longer. Alternatively, other suitable adhesives, tacky, or non-slip materials may be provided for the adhesive region 28, e.g., to fix the device 10 to the patient's skin with or without other attachment devices.

In one embodiment, the adhesive region 28 may extend substantially continuously around the periphery of the patient contact surface 22. Alternatively, adhesive or other tacky material may be provided intermittently or discontinuously around and/or otherwise on the periphery and/or may be configured in various geometric patterns, such as radial or concentric stripes and the like (not shown), to provide adequate adhesion to the patient's skin to prevent unintentional removal of the device 10.

In another alternative, the gel reservoir 26 may be omitted and the entire patient contact surface 22 of the device 10 may be substantially flat or otherwise shaped to be placed against the patient's skin (not shown). In this alternative, the entire patient contact surface may be covered with adhesive, tacky material, and/or coupling material, rather than only around the periphery (not shown). Without the gel reservoir, adhesive material alone may provide sufficient acoustic coupling to the patient's skin. For example, double stick adhesives may be used, e.g., with a first surface permanently attached to the patient contact surface 22 of the device 10 and a second surface exposed for placement against the patient's skin to provide acoustic coupling between the skin and the acoustic transducer 30 of the device 10, as well as substantially secure fixation.

The device 10 may be provided with a peel-away cover, release paper, package enclosure, and the like (not shown), which may protect the patient contact surface 22, e.g., to prevent premature exposure of the adhesive region 28 and/or coupling material if provided within the gel reservoir 26, before application of the device 10 to a patient's skin. The cover or enclosure may be removed to expose the adhesive region 28 and/or coupling material immediately before the patient contact surface 22 is applied to a patient's skin, as described elsewhere herein.

With particular reference to FIG. 2A, within the housing 20, the device 10 may include an acoustic transducer 30 acoustically coupled to the patient contact surface 22, e.g., adjacent the gel reservoir 26. For example, the acoustic transducer 30 may be provided directly above the gel reservoir 26 and/or may be separated from the gel reservoir 26 by a thin layer of encasement material 29, as shown, e.g., having a thickness between about 0.125 to 3.1 millimeters (0.005-0.125 inch) thick. The thin layer of encasement material 29 may transmit substantially all incident and reflected acoustic energy passing therethrough, e.g., to reduce energy loss through the encasement material 29. Alternatively, there may be no encasement material between the gel reservoir 26 and the lower surface 32 of the acoustic transducer 30 may form a ceiling for the gel reservoir 26, thereby directly coupling the exposed surface 32 of the acoustic transducer 30 to acoustic coupling material within the gel reservoir 26 (not shown). In this alternative, a thin, solid gel pad layer may be provided, e.g., molded onto the lower surface 32 of the acoustic transducer 30. Optionally, an internal reservoir of aqueous gel or other coupling material may be provided that may release slowly into the solid gel pad layer, e.g., via one or more channels (not shown), to provide acoustic coupling over an extended period of time, e.g., days or weeks.

One or more processing circuits or other processors 40, e.g., one or more printed circuit boards with integrated circuits, ASICs, and/or other hardware components (not shown), may be provided within the housing 20 capable of driving the acoustic transducer 30 and/or receiving signals from the acoustic transducer 30. For example, the processor 40 may include a driver circuit for activating the acoustic transducer 30 to transmit acoustic energy, and a processor circuit for analyzing reflected acoustic energy received by the acoustic transducer 30, as described further elsewhere herein. Alternatively, if high resolution images are not needed, the driver circuit may be omitted, which may reduce overall power consumption and/or reduce the thickness or other profile of the device 10.

In addition, a power source 50, such as a lithium ion battery and the like, may be provided within the housing 20 capable of providing electrical power to the processor(s) 30, acoustic transducer 40, and/or other components of the device 10. The power source 50 may provide sufficient energy to the components to operate the device 10 for its desired life, e.g., several days, whereupon the entire device 10 may be discarded or replaced with another device, as desired. Alternatively, the power source 50 may be rechargeable, e.g., from an external power source (not shown), if desired, to extend the life of the device 10. In a further alternative, the housing 10 may include a port (not shown) that may be opened to access and replace a depleted power source 50 with a fresh one, if desired. In yet a further alternative, the housing 20 may include a connector (not shown) for coupling the components to an external power source (also not shown), and the internal power source 50 may be omitted.

As shown in FIG. 2A, the processor(s) 40 and/or power source 50 may be placed above the acoustic transducer 30 within the housing 20, e.g., between the acoustic transducer 30 and the outer surface 24 of the housing 20, which may reduce acoustic energy being dispersed away from the patient contact surface 22 and the patient's body. In addition or alternatively, a separate acoustic backing material or layer (not shown) may be included between the components, e.g., between the acoustic transducer 30 and the outer surface 24 of the housing 20, to enhance directing acoustic energy from the acoustic transducer 30 towards the patient contact surface 22 and the patient's body.

The acoustic transducer 30 may have sufficient surface area to cover a desired area of a patient's body, e.g., having a desired width and sufficient length to overlap a plurality of rib bones of a patient when the device 10 is applied transversely relative to the patient's ribs, e.g., as shown in FIG. 7 and described further elsewhere herein. In an exemplary embodiment, the acoustic transducer 30 may have a length between about nineteen and seventy five millimeters (0.75-3.0 inches), which may correspond to the overall length of the device 10. For example, the housing 20 may have an outer length between about twenty five and one hundred millimeters (1.0-4.0 inches) to accommodate such an acoustic transducer 30.

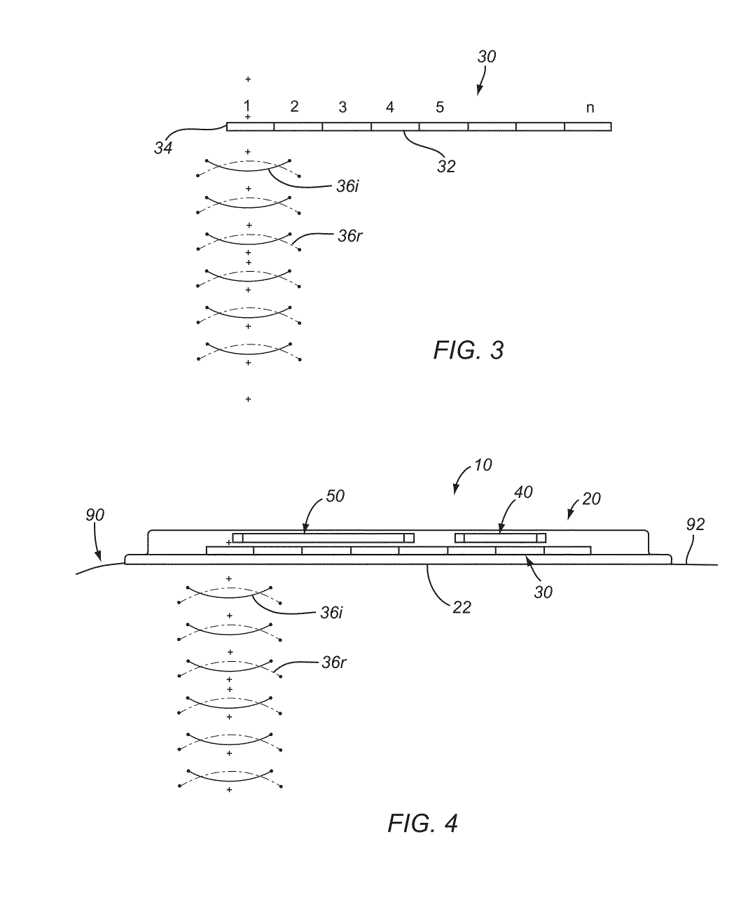

Turning to FIGS. 3 and 4, an exemplary embodiment of a transducer array is shown that may be provided for the acoustic transducer 30. As shown, the acoustic transducer 30 may include a plurality of transducer elements 34 aligned in a row, e.g., such that the length of the transducer array extends between or over two or more ribs of a patient's thorax. For example, the acoustic transducer 30 may be a piezoelectric transducer that includes a plurality “n” of single and individual piezoelectric elements 34, each capable of producing a focused beam of acoustic energy.

Current piezoelectric ultrasound probes typically use large arrays including one hundred twenty eight (128) elements or more, which may be activated simultaneously or individually. These may provide high resolution images but are very expensive and consume substantial power, thereby requiring a large external power source, in contrast to a small power source 50, which may be used in the devices 10 and systems described herein. Advantages of a series of single, individually focused elements may include reduction in cost, size, and/or power consumption, as compared to such large, high resolution acoustic imaging devices. In an exemplary embodiment, the acoustic transducer 30 may include between about one and sixteen (n=1-16) transducer elements 34, i.e., a significantly fewer number of elements than other ultrasound systems, which reduce the power consumption of the device 10 and/or increase its active life.

As shown in FIGS. 3 and 4, the individual elements 34 may be configured as a strip with a single transducer element 34 across the width of the acoustic transducer 30 and a plurality of “n” transducer elements 34 having substantially the same length and spaced apart substantially uniformly along the length of the acoustic transducer 30. As shown schematically in FIG. 3, the acoustic transducer 30 may be activated such that the surface of a first transducer element 34(1) transmits incident acoustic energy 36i and receives reflecting acoustic energy 36r, e.g., from a respective adjacent region of the thorax. For example, the processor 40 may activate the transducer elements 34 individually, i.e., sequentially or otherwise alternately, e.g., 34(1) first, 34(2) second, . . . to 34(n) cyclically, to transmit and receive acoustic energy, which the processor 40 may then analyze to monitor extravascular lung water or pulmonary edema, as described elsewhere herein.

In the case of a piezoelectric transducer, the acoustic transducer 30 may be configured to transmit acoustic energy at a frequency between about five and fourteen Megahertz (5-14 MHz). Alternatively, the acoustic transducer 30 may be a capacitive micromachined ultrasound transducer (“CMUT”), such as those described in U.S. Pat. No. 5,619,476, the entire disclosure of which is expressly incorporated by reference herein. In the case of a capacitive micromachined ultrasound transducer, the acoustic transducer 30 may be configured to transmit acoustic energy at a frequency between about 1.5 and five Megahertz (1.5-5 MHz). In a further alternative, the material used for the transducer may be a polymer with piezoelectric properties, such as polyvinylidene fluoride, which may provide greater surface area coverage at relatively lower cost compared to other materials.

In another alternative embodiment, the acoustic transducer 30 may include a single crystal ultrasound transducer element. Compared with piezoelectric materials and CMUT-based transducers, single crystal materials and composites may be well suited for small, low power, long term applications, such as the acoustic diagnostic devices herein. They may have better acoustic impedance matching, which is useful for a long-term wearable device that relies on gel or silicone pad for impedance matching. In addition, they may be configured to operate at a frequency between about three and five Megahertz (3.0-5.0 MHz) and may include relatively large transducer elements, which may be useful for determining ultrasound B-lines in a patient's body. Exemplary embodiments of crystal transducer elements that may be used are disclosed in Lu, X. M., Proulx. T. L., Single crystals vs. PZT ceramics for medical ultrasound applications, 2005 IEEE Symposium, pp. 227-230 and Rhim, S. M., Jung, H., Piezoelectric Single Crystal for Medical Ultrasound Transducer, 2007 IEEE Symposium, pp. 300-304, the entire disclosures of which are expressly incorporated by reference herein.

During use, as shown in FIGS. 4 and 7, the device 10 may be attached or otherwise worn by a patient 90, e.g., a patient who may be at risk for extravascular lung water, such as those suffering from heart failure. It will be appreciated that the devices herein may be used for other diagnostic applications in which acoustic images of a patient's body may be acquired and analyzed, e.g., other lung conditions, such as alveolar-interstitial fluid, pulmonary consolidation, pleural effusion, pneumothorax, hemothorax, chylothorax, and the like.

For example, as best seen in FIG. 4, the patient contact surface 22 of the device 10 may be adhered and/or otherwise fixed to the skin 92, e.g., to a patient's thorax at a location that allows imaging of the left lung, right lung, or both. For example, if provided with a cover or package (not shown), the cover or package may be removed to expose the patient contact surface 22, e.g., the adhesive region 28 and/or gel reservoir 26, and the adhesive region 28 (not shown, see FIG. 2A) may be adhered to the skin 92 with coupling material (not shown) within the gel reservoir 26 coupling the acoustic transducer 30 to the patient's skin 92. Optionally, e.g., similar to the embodiment shown in FIG. 7, the thorax may be divided into separate regions and multiple devices 10 may be placed at various locations. In an exemplary embodiment, the device(s) 10 may be placed at lateral locations along the patient's side, which may have the highest correlations with instances of pulmonary edema.

If the gel reservoir 26 is provided initially with coupling material, the coupling material may contact and/or otherwise acoustically couple the device 10 to the patient's skin 92 and underlying tissue. If the gel reservoir 26 is provided empty, ultrasound gel or other coupling material may be applied into the empty gel reservoir 26 and then the adhesive region 28 of the device 10 may be affixed to the patient's skin 92. Alternatively, if the device 10′ of FIG. 2B is provided, gel or other coupling material may be injected through the injection holes 27′ into the gel reservoir 26,′ e.g., before or after fixing the device 10′ to the patient 90.

Optionally, the device 10 may be fixed relative to the patient's body 90 using other external devices, such as a strap, band, tape, and the like (not shown), e.g., in addition to or instead of the adhesive region 28 on the patient contact surface 22. For example, one or more straps may be received through loops (not shown) on either end of the housing 20 and wrapped around the patient's torso and/or otherwise secured in place. In addition or alternatively, adhesive tabs or other extensions (not shown) may be provided, e.g., extending from each end of the housing 20, which may provide additional adhesive or other fixation material to secure the device 10 to the patient's skin, similar to a bandage.

Once fixed, the device 10 may be used to monitor the patient's extravascular lung water status. For example, once secured, the device 10 may be activated, e.g., by pressing an “on/off” button or other actuator 69 (not shown, see, e.g., FIG. 1B or 7). Once activated, the processor 40 may control and/or communicate with the acoustic transducer 30, e.g., to transmit and/or receive acoustic energy. For example, as shown in FIG. 4, the processor 40 may direct the acoustic transducer 30 to emit transmitting or incident acoustic signals or energy 36i into the patient's body 90, e.g., past the ribs towards the lung(s). As the incident acoustic energy 36i passes through various tissues, acoustic echoes are created and reflected back toward the acoustic transducer 230, as represented by reflected acoustic signals or energy 36r shown in FIG. 4.

The reflected signals 36r may be communicated to the processor 40, which may interpret the acoustic echoes to determine and/or analyze the extravascular lung water status of the patient 90. For example, if extravascular lung water is present in the intralobular spaces of a lung receiving the incident signals 36i, the acoustic echoes 36r that return to the acoustic transducer 30 may appear as ultrasound B-lines, i.e., sustained reflections over time. The processor 40 may calculate the frequency, intensity, width, and/or other properties of these sustained acoustic echoes 36r, e.g., over a predetermined period of time to monitor the patient's extravascular lung water status.

Imaging of ultrasound B-lines may allow for the quantification of the B-lines, e.g., determining an actual number of B-lines detected and/or the intensity and/or width of the B-lines. For example, by counting the intensity of the B-lines, the processor 40 may provide an accurate measure of the fluid status by effectively counting the amount of “white space,” e.g., as seen by a traditional ultrasound imaging technique.

In an exemplary embodiment, the processor 40 may divide the reflected acoustic echoes or images into sections, e.g., “pixels” (even though no actual image may actually be displayed) and determine whether the intensity of individual sections exceed a predetermined brightness threshold, which is determined based on the intensity of the acoustic energy received by the acoustic transducer 30. If so, the identified sections may be considered “white space” and the number of such “white space” sections as a function of the total number of sections may be monitored, e.g., as a percentage or other value, over time to detect changes in the amount of extravascular lung water present. For example, if the percentage of “white space” increases over time, e.g., from substantially continuous or discontinuous images, the processor 40 may determine that the amount of extravascular lung water within the patient's thorax is increasing. Such outcomes and/or trends may be stored by the processor 40 for subsequent analysis and/or may be communicated to other devices, as described elsewhere herein. It should be understood that although the language used to describe the function of the analysis refers to an image with black space (where there are no B-Lines) and white space (where there are B-Lines), that no actual image may be collected but that these characteristics may still be gathered from the reflected echoes received by the acoustic transducer 30 and analyzed by the processor 40.

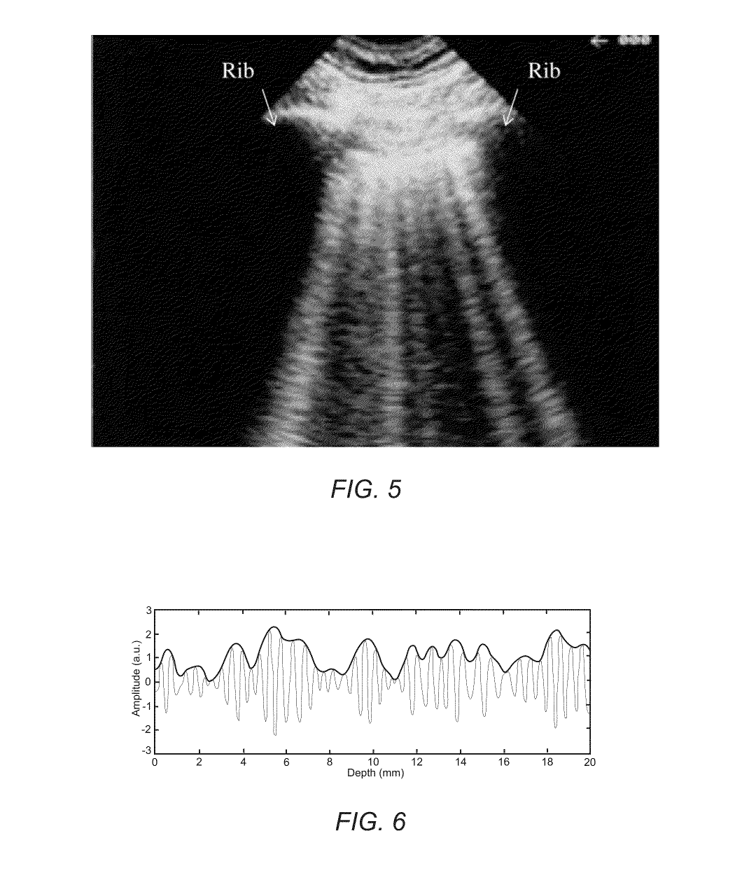

In addition or alternatively, the device 10 may be placed over one or more ribs such that the acoustic transducer 30 transmits acoustic energy 36i towards the rib(s). Bones provide poor reflection of acoustic energy and as such are identifiable from other thoracic tissue. The processor 40 may use the rib(s) as a landmark to monitor the ultrasound B-lines in substantially the same location over time. For example, FIG. 5 shows an exemplary ultrasound image in which two ribs may be identified as dark regions on either side of the B-lines. In this example, the processor 40 may monitor the intensity of the B-lines between the ribs over time to monitor the extravascular lung water status of the patient. If the patient moves, thereby shifting the device 10 on their skin slightly, the processor 40 may use the ribs as landmarks to identify the region that is being monitored between them, even if the ribs move slightly within the field of the acoustic echoes or images.

The device 10 may substantially continuously monitor the thorax, e.g., for periods of days or weeks, e.g., until the actuator 60 is pressed to deactivate the device 10. Alternatively, the device 10 may be activated manually for discrete periods of time. For example, when the actuator 60 is pressed, the device 10 may be activated only for sufficient time to determine the current extravascular lung water status, whereupon the device 10 may automatically shut off or hibernate, which may be beneficial from a power management perspective.

In a further alternative, the device 10 may monitor the patient at discrete, e.g., periodic or other intermittent, intervals over an extended period of time, e.g., including one or more days. For example, the device 10 may be configured to activate the acoustic transducer 30 to transmit and receive acoustic energy in the morning, at mid-day, and in the evening of each day, e.g., at preset times.

Optionally, the device 10 may include one or more accelerometers and/or other motion sensors 62 (not shown, see FIG. 2B), e.g., within the housing 20 and coupled to the processor 40, which may determine the movement and/or body position of the patient based on data from the motion sensor(s) 62. In this embodiment, the processor 40 may only activate the acoustic transducer 30 to transmit and receive acoustic energy during predetermined motion states, e.g., when the patient is lying down or only during periods of non-movement, thereby increasing consistency between serial measurements. For example, if a scheduled time of activation is determined by the processor 40, the processor 40 may obtain data from the motion sensor(s) 62 to confirm the patient's activity status and/or position. If the desired status (e.g., inactive or prone) is not confirmed, the processor 40 may delay activation for a predetermined time until the desired status is finally confirmed, whereupon the processor 40 may activate the acoustic transducer 30. Such a configuration may reduce the electrical power consumed by the device 10 (e.g., since data may be acquired only during desired time periods when a desired activity status is confirmed) and/or may reduce the overall size of the device 10, e.g., by reducing the size of the power source 50 needed.

With continued reference to FIGS. 1B and 7, optionally, the device 10 may include one or more output devices 70, e.g., a display or other visual indicator, a speaker or other audible indicator, and the like. In exemplary embodiments, the output device 70 may include one or more of an LCD or other display, a series of LED lights, an alert or alarm, and the like, e.g., to notify the patient and/or their caregiver of a worsening or otherwise changed condition.

For example, as shown in FIG. 7, the output device 70 may be a set of indicator lights, which may provide a simple output of the patient's current extravascular lung water status. For example, a first indicator (e.g., a green light) may be activated by the processor 40 when the patient's extravascular lung water status is below a predetermined threshold. If the processor 40 determines that patient's status has changed, i.e., the patient's extravascular lung water is increasing, a second indicator (e.g., a yellow light) may be activated to provide a visual indication of the change. If the processor 40 determines that the patient's extravascular lung water has increased beyond a predetermined threshold, a third indicator (e.g., a red light) may be activated. Thus, visual indications may be provided to guide the patient 90 and/or their caregivers.

In addition or alternatively, the device 10 may include a communication interface 64, e.g., a wireless transmitter and/or receiver (not shown, see FIG. 1B) within the housing 20 and coupled to the processor 40 for transmitting information regarding the patient's status to a remote location. In an exemplary embodiment, the communication interface 64 may include a radio-frequency transmitter, e.g., using Bluetooth or other protocols, to communicate information wirelessly to a base station 102, as shown in FIG. 7 (which may itself include a corresponding communication interface, not shown). For example, the processor 40 may communicate extravascular lung water data for the patient 90 substantially continuously, periodically, and the like, e.g., each time the acoustic transducer 30 is activated, or the processor 40 may accumulate data in memory (not shown), and only transmit data periodically or when there is a predetermined change, e.g., increase or rate of increase in extravascular lung water.

The base station 102 may record the extravascular lung water status of the patient 90 over time, e.g., when data is received from the device 10. In addition or alternatively, the communication interface 64 of the device 10 may include a receiver for communicating commands from the base station 102 to the processor 40, e.g., as represented by signals 104. For example, rather than the processor 40 intermittently activating the acoustic transducer 30, the processor 40 may simply wait for a command communicated by the base station 102 via the communication interface 64 to do so, whereupon the processor 40 may activate the acoustic transducer 30 and communicate the resulting information back to the base station 102. In addition or alternatively, the base station 102 may communicate with multiple devices 10 fixed to the patient 90, e.g., the two devices 10a, 10b shown in FIG. 7, thereby providing a system 100 for monitoring the patient 90.

During operation, for example, if the absolute level of extravascular lung water of the patient 90 reaches a given threshold or if the rate of extravascular lung water accumulation (e.g., percentage increase over time) reaches a given threshold (from one device 10 or multiple devices 10a, 10b), the base station 102 may include an alarm, signal generator, or other output device (not shown) to alert the patient 90 and/or their caregiver of the condition. In addition or additionally, the base station 102 may include a network communication interface, e.g., a wireless interface, telecommunication interface, and the like (not shown) capable communicating via a network (also not shown), as represented by signals 106. For example, the network interface may communicate over a telecommunications network and/or the Internet, to provide information from the device 10 to a healthcare provider, e.g., such that the healthcare provider may make clinical decisions based on the information. For example, if the patient 90 is receiving care, the provider may determine that diuretics and/or other medications should be administered to the patient 90, or that the patient 90 should admitted to a hospital (e.g., if being monitored remotely from home).

Additionally, as shown in FIG. 7, if desired, a plurality of devices 10 (two devices 10a, 10b shown merely for example) may be placed on the patient 90, e.g., at different locations of the thorax. In this case, the base station 102 may communicate with each device 10 and then determine the extravascular lung water status from data from individual devices 10 or based on a combination of the collective data from each device 10.

Alternatively, a first device 10a may be configured only to transmit acoustic energy (not shown) and a second, separate device 10b may be configured only to receive acoustic echoes (also not shown). In this case, the devices 10a, 10b may work in conjunction across different sections of the thorax to determine the extravascular lung water status of the patient 90. A single pair or multiple pairs (not shown) of such devices may be provided, as desired, with the base station 102 receiving, analyzing, and/or communicating the resulting information.

In another alternative embodiment, a plurality of devices 10 may be provided, with the acoustic transducer 30 of each device 10 including only a single piezoelectric (or other transducer) element (not shown). In this alternative, the size of each device may minimized further and/or the power consumption of each device may be reduced. In this embodiment, the plurality of devices may be fixed at several locations on the patient 90 and then used by a processor on a master device (receiving data from other devices) or by a base station 102, to provide an indication of the extravascular lung water status of the patient 90.

Optionally, in any of these embodiments, one or more devices 10 may be secured to a patient 90 using a removable accessory such as a chest band or strap (not shown), e.g., instead of or in addition to an adhesive region on each device 10. For example, a chest band may be provided that includes multiple devices 10 or multiple acoustic transducers (not shown) spaced apart along the band in a desired manner, e.g., such that the acoustic transducers may be positioned at various locations along the thorax when the chest band is secured around the patient 90. The chest band may be an elastic strap that applies a radially inward force between the devices 10 and the patient's skin 92, e.g., to hold and/or otherwise couple the acoustic transducers against the skin of the thorax. In this embodiment, the chest band may be worn substantially continuously throughout the day and night by the patient, or only during discrete intervals throughout the day, e.g., when the patient's status is scheduled to be determined.

Alternatively, one or more device(s) 10 may be provided that do not include an internal power source and/or processor. For example, a power source and/or processor may be provided in a base station, such as the base station 102 shown in FIG. 7, and the device(s) 10 may only be operated when connected to the base station 102, e.g., by one or more cables, a wireless communication interface, and the like. In this embodiment, the device(s) 10 may remain adhered or otherwise coupled to the thorax and, at discrete times, the base station 102 may be connected to or otherwise communicate with the device(s) 10, e.g., wirelessly or through a direct connection, to activate the acoustic transducer(s) and communicate resulting data to the base station 102. During these connection periods, the base station 102 may provide power to the device(s) 10, e.g., to transmit and/or receive acoustic energy. The received acoustic echoes may then be communicated to and analyzed by the base station 102. In this system (or other system including a base station 102), the patient 90 may be instructed to move into a posture or series of postures, e.g., pre-determined or guided by the base station 102.

In another alternative, instead of a substantially continuously wearable device, an acoustic diagnostic device may be provided that is incorporated into a handheld system (not shown). For example, a handheld device may be provided that includes a patient contact surface coupled to an acoustic transducer (not shown) such that the patient contact surface may be held against the patient's skin, e.g., against the thorax at a desired location to transmit and receive acoustic energy to monitor the patient's extravascular lung water status. Optionally, for such a system, one or more desired locations may be marked on the patient's body, e.g., using non-permanent ink and the like, to ensure the same location(s) are used for a series of readings. The handheld device may include a processor (not shown) that receives the acoustic echoes and uses an algorithm, e.g., identifying anatomical landmarks, for example, the ribs, to confirm that the same location is used for subsequent readings. Such an algorithm may allow the device to be used by the patient him/herself, and the data and/or analysis may be displayed or communicated to a remote location, similar to other embodiments herein. Thus, the patient may only need to place the patient contact surface of the device approximately in a desired location, and the algorithm may automatically identify the correct area to scan.

In still another alternative embodiment, all or part of the device (e.g., the transducer elements) may be implanted subdermally or subcutaneously. If the transducer elements are implanted under the skin, which may provide a smaller form factor, an external portion (e.g., handheld) device may be used for any or all of the following functions: (1) providing a signal to the implantable portion to scan the patient, (2) wirelessly charging a battery or other power source of the implantable portion during use or during sleep, (3) serving as a data receiver and analyzer for the implantable portion, (4) transmitting the data to a remote location, e.g., to a remote server via a mobile internet connection, or to a local device (such as a desktop device, base station, and the like), which may then communicate the information to the patient, caregivers, or clinical support personnel.

Turning to FIG. 6, the devices herein may be used to determine extravascular lung water status of a patient using ultrasound RF-signal phase information, e.g., instead of or in addition to analyzing acoustic echoes. As described elsewhere herein, ultrasound B-lines are generated based on reverberations of water and air in the interstitial and alveolar spaces of the lung. To gain additional sensitivity, phase information from the raw RF-signal (typically lost during normal envelope detection, as shown in FIG. 6) may be used to differentiate between normal lung tissue and lung tissue with pulmonary edema.

Although such analysis has been used to differentiate between tissue types in other applications, devices herein, such as the device 10 of FIGS. 1A and 2A may use the phase information to differentiate between “normal” lung tissue and lung tissue with extravascular fluid. A wide band signal may be applied by the acoustic transducer 30, and the processor 40 may analyze the reflected signals to identify reverberations at specific frequencies, phases, phase differences, and/or other statistical properties of the reflected signal that correspond to increased fluid. Alternatively, the processor 40 may scan through a range of frequencies and analyze the frequency response curve in the reflected signals to identify the amount of extravascular lung water present. Echoes with higher frequencies in the frequency response curve may represent significantly increased reverberations, representing more fluid-filled pockets and thus signifying increased extravascular lung water.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A wearable acoustic diagnostic device, comprising:
a housing configured to be worn by a patient and including a patient contact surface configured to contact the patient's skin of the patient's thorax;
an acoustic transducer for transmitting acoustic energy via the patient contact surface into the patient's thorax and receiving reflected acoustic energy from the patient's thorax; and
one or more processors coupled to the acoustic transducer for analyzing the reflected acoustic energy to provide an indication of extravascular lung water status of the patient.

2. The device of claim 1, wherein the patient contact surface comprises a periphery surrounding a gel reservoir, the periphery comprising an adhesive region to substantially fix the housing relative to the patient's skin.

3. The device of claim 2, further comprising acoustic coupling material in the gel reservoir for acoustically coupling the acoustic transducer to the patient's skin.

4. The device of claim 1, further comprising means for securing the housing to the patient's thorax.

5. (canceled)

6. The device of claim 1, wherein the acoustic transducer comprises a linear array including a plurality of transducer elements.

7. The device of claim 6, wherein the linear array has a length sufficient to overly two or more ribs of a patient.

8-10. (canceled)

11. The device of claim 1, wherein the one or more processors are configured to analyze the reflected acoustic energy to determine a number of the responsive acoustic echoes per unit of time.

12. The device of claim 1, wherein the one or more processors are configured to analyze the reflected acoustic energy to determine at least one of an intensity and a frequency of the reflected acoustic energy.

13. The device of claim 1, wherein the acoustic transducer is positioned between the one or more processors and the patient contact surface such that the one or more processors provide an acoustic backing layer to enhance transmission of acoustic energy from the acoustic transducer through the patient contact surface towards the patient's thorax.

14. The device of claim 1, further comprising an acoustic backing layer adjacent the acoustic transducer to enhance transmission of acoustic energy from the acoustic transducer through the patient contact surface towards the patient's thorax.

15. The device of claim 1, further comprising a power source within the housing coupled to at least one of the one or more processors and the acoustic transducer.

16. The device of claim 15, wherein the acoustic transducer is positioned between the power source and the patient contact surface such that the power source provides an acoustic backing layer to enhance transmission of acoustic energy from the acoustic transducer through the patient contact surface towards the patient's thorax.

17. The device of claim 1, wherein the one or more processors are configured to intermittently activate the acoustic transducer to transmit acoustic energy via the patient contact surface into the patient's thorax and receive reflected acoustic energy from the patient's thorax, and wherein the one or more processors analyze the reflected acoustic energy to determine changes in the extravascular lung water status of the patient over time.

18-19. (canceled)

20. The device of claim 1, further comprising a motion sensor coupled to the one or more processors, and wherein the one or more processors are configured to acquire motion data from the motion sensor to determine an activity status of the patient, and wherein the one or more processors activate the acoustic transducer only when a predetermined activity status of the patient is confirmed.

21. The device of claim 1, further comprising an output device coupled to the one or more processors for providing an indication of the extravascular lung water status of the patient.

22-24. (canceled)

25. A system for monitoring extravascular lung water of a patient, comprising:
a) an acoustic diagnostic device, comprising:
i) a housing configured to be worn by a patient and including a patient contact surface configured to contact the patient's skin of the patient's thorax;
ii) an acoustic transducer for transmitting acoustic energy via the patient contact surface into the patient's thorax and receiving reflected acoustic energy from the patient's thorax;
iii) one or more processors coupled to the acoustic transducer for processing the reflected acoustic energy; and
iv) a communication interface coupled to the one or more processors for communicating information regarding the reflected acoustic energy to a remote location; and
b) a base station for receiving the information via the communication interface, and monitoring an extravascular lung water status of the patient based at least in part on the information.

26. (canceled)

27. The system of claim 25, wherein the base station comprises a processor configured to analyze the information regarding the reflected acoustic energy to determine a number of the responsive acoustic echoes per unit of time.

28. The system of claim 25, wherein the base station comprises a processor configured to analyze the information regarding the reflected acoustic energy to determine at least one of an intensity and a frequency of the reflected acoustic energy.

29. The system of claim 25, wherein the communication interface comprises a receiver for receiving instructions from the base station, the base station configured to send instructions to the acoustic diagnostic device to intermittently activate the acoustic transducer to transmit acoustic energy and receive reflected acoustic energy from the patient's thorax, and wherein the base station is configured to analyze the reflected acoustic energy to determine changes in the extravascular lung water status of the patient over time.

30-32. (canceled)

33. A method for monitoring extravascular lung water of a patient, comprising:
fixing an acoustic transducer device relative to the patient's skin of the patient's thorax;
activating the acoustic transducer device to transmit acoustic energy into the patient's thorax and receive reflected acoustic energy from the patient's thorax; and
analyzing the reflected acoustic energy to monitor the extravascular lung water status of the patient.

34-59. (canceled)
